(19) United States
(12) Patent Application Publication
(10) Pub. No.: US 2005/0203638 A1
(43) Pub. Date: Sep. 15, 2005

(54) Modular stance flexion component for a prosthetic limb

(76) Inventors: Tracy C. Slemker, Clayton, OH (US); Scott R. Schall, Elnglewood, OH (US); Lucinda Busch, Clayton, OH (US); Steven Steinbarger, Wilmington, OH (US); Robert Hoskins, Dayton, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 11/069,832

(22) Filed: Mar. 1, 2005

Related U.S. Application Data
(60) Provisional application No. 60/549,115, filed on Mar. 1, 2004.

(51) Int. Cl. A61F 2/60

(52) U.S. Cl. 623/35

(57) ABSTRACT

A modular prosthetic limb component is adapted to be selectively/removably interconnected between a prosthetic knee component and a prosthetic socket component, where the modular component provides or augments stance flexion approximate the proximate knee component.

### CROSS REFERENCE TO RELATED APPLICATIONS

This application claims the benefit of U.S. Provisional Patent Application No. 60/549,115, filed Mar. 1, 2004, the disclosure of which is incorporated herein by reference.

### BACKGROUND

Stance Flexion is a term used in the prosthetic industry to describe a feature on a knee-shin system that allows the amputee to exhibit “bent knee walking.” In a more simplistic form the stance flexion feature allows the knee axis to bend slightly under weight bearing without comprising the stability of the knee. Compromised knee stability can cause an amputee to break the knee and stumble. Stance flexion allows a certain degree of knee flexion at heel strike to more closely mimic normal gait patterns for non-amputees. This also acts as a shock absorbing component that may reduce the forces felt by the amputee's residual limb during ambulation.

Advantageously, there exists an established HCPC code for a Stance Flexion feature that states: Addition to an endoskeletal knee-shin system, stance flexion feature, adjustable. AOPA interpretation: An adjustable addition to a knee-shin system that allows the knee to flex slightly under weight-bearing forces, to absorb shock, conserve energy, and normalize gait.

Presently, there is no known modular stance flexion component on the market. All known stance flexion features are presently built into the knee-shin system itself. They are adjustable and work reasonably well. However, a modular adjustable component is desired so that prosthetists may use this feature on a majority of the knees available without the stance flexion feature, thereby increasing the number of amputees who can benefit from this feature.

### SUMMARY

It is a first aspect of the present invention to provide a modular prosthetic limb component adapted to be selectively/removably interconnected between a prosthetic knee component and a prosthetic socket component, where the modular component provides (or augments) stance flexion approximate the proximate knee component.

It is a second aspect of the present invention to provide a modular prosthetic limb component adapted to be selectively/removably interconnected between a prosthetic knee component and a prosthetic socket component, where the modular component comprises: (a) a first vertical end adapted to be coupled to a first prosthetic limb component, (b) a second vertical end adapted to be coupled to a second prosthetic limb component, (c) an anterior pivot point operatively coupled between the first and second vertical ends which allows the first and second prosthetic limb components to pivot inwardly with respect to each other, and (d) a bias operatively provided between the first and second vertical ends which allows the first and second prosthetic limb components to flex towards each other approximate the posterior end thereof at least under weight bearing forces and/or heel strike.

It is a third aspect of the present invention to provide a modular prosthetic limb component adapted to be selectively interconnected between a prosthetic knee component and a prosthetic socket component, where the modular component comprises a substantially C-shaped body (having a pair of substantially horizontal top and bottom extensions emanating from a curved end) of adequately flexible and strong material (such as, for example, a carbon fiber material), where the extensions of the C-shaped body each include couplings for coupling to other prosthetic limb components (i.e., the top extension adapted to be coupled to a proximal prosthetic limb component and the bottom extension adapted to be coupled to a distal prosthetic limb component), where the curved end of the C-shaped body is adapted to be positioned approximate the anterior of the prosthetic limb knee component and where the open end of the C-shaped body is adapted to be positioned approximate the posterior of the prosthetic knee component, and where the modular component further comprises a posterior bias/shock-absorber (such as, for example, a rubber-like sphere) operatively provided between the top and bottom extensions. With at least this third aspect, it is within the scope of the invention that the modular component is able to be flipped over if desired (i.e., the top extension becomes the bottom extension adapted to be coupled to a distal prosthetic limb component and vice-versa), while keeping the curved end positioned approximate the anterior of the prosthetic limb knee component. This ability to be flipped over provides the modular component flexibility for interconnecting a broader range of prosthetic limb components.

With any of the above three aspects, it is further within the scope of the invention to provide a posterior retainer (such as a strap) that substantially limits posterior outward expansion (i.e., provides a hyperextension limit) of the component.

A fourth aspect of the invention provides a method for providing stance flexion in a prosthetic limb that includes the step of selectively/removably coupling a modular stance flexion component (such as described above) between a prosthetic limb socket component and a prosthetic limb knee component.

A fifth aspect of the invention modifies the purpose somewhat in that it is provided primarily as a modular shock absorber component for a prosthetic limb. In this forth aspect, a modular prosthetic limb component adapted to be selectively interconnected between two prosthetic limb components comprises a body of adequately flexible and strong material (such as, for example, carbon fiber material) substantially in the shape of a vertically compressed “O” (having a pair of opposing, substantially flat horizontally extending top/bottom ends interconnected by a pair of opposing, substantially curved anterior/posterior ends), where each of the flat horizontally extending top/bottom ends of the body include couplings adapted to be coupled to a respective one of the two prosthetic limb components, and where each of the curved anterior/posterior ends allow the top/bottom ends to flex together at least upon moments of impact such as heel strike. With this fourth aspect, it is also within the scope of the invention that the modular component is able to be flipped over if desired (i.e., the top extension becomes the bottom extension adapted to be coupled to a distal prosthetic limb component and vice-versa).

With any of the above aspects, it is also within the scope of the invention to integrate such components into a proximal end of a prosthetic knee chassis (such as a prosthetic knee-shin system). In such an embodiment, the component would no longer be modular (i.e., selectively coupled) with respect to the knee chassis.

Furthermore, with any of the above aspects of the invention, it is within the scope of the invention that one or more of the coupling expedients include, without limitation: an integrated pyramid coupling (which may or may-not be rotatably and/or laterally adjustable), an integrated pyramid receiver (which may or may-not be rotatably and/or laterally adjustable), one or more sets of threaded holes (i.e., in the standard four-hole pattern), or one or more sets of bolts/screw receiving through holes or bores.

### BRIEF DESCRIPTION OF THE DRAWINGS

FIG. 1 is a side, elevational view of a modular stance flexion component according to an exemplary embodiment of the present invention;

FIG. 2 is a perspective view taken from a posterior end of the modular stance flexion component of FIG. 1;

FIG. 3 is a perspective view taken from an anterior end of the modular stance flexion component of FIGS. 1 and 2;

FIG. 4 is a view of a bottom/top face of the modular stance flexion component of FIGS. 1-3;

FIG. 5 is an exploded view of a prosthetic limb assembly incorporating the modular stance flexion component of FIGS. 1-4;

FIG. 6 is an elevational side view of an alternative embodiment of the modular stance flexion component according to the present invention;

FIG. 7 is an elevational side view of an another alternative embodiment of the modular stance flexion component according to the present invention;

FIG. 8 is an exploded view of a prosthetic limb assembly illustrating a modular stance flexion component according to the present invention integrated into a proximal end of a knee chassis component; and

FIG. 9 illustrates an embodiment of a modular shock absorbing component according to an embodiment of the present invention.

### DETAILED DESCRIPTION

The present invention provides a modular prosthetic limb component that is adapted, in an exemplary embodiment, to be selectively (i.e., removably) interconnected between a prosthetic knee component and a prosthetic socket component (adapted to receive the patient's residual limb), where the modular component provides (or augments) stance flexion approximate the prosthetic knee component. In a more detailed exemplary embodiment, the invention provides a “C” shaped (in elevational cross-section) component of carbon fiber or some other resilient, lightweight, high strength material that will allow a degree of compression upon weight bearing. The “adjustability” criteria may be provided by various design applications.

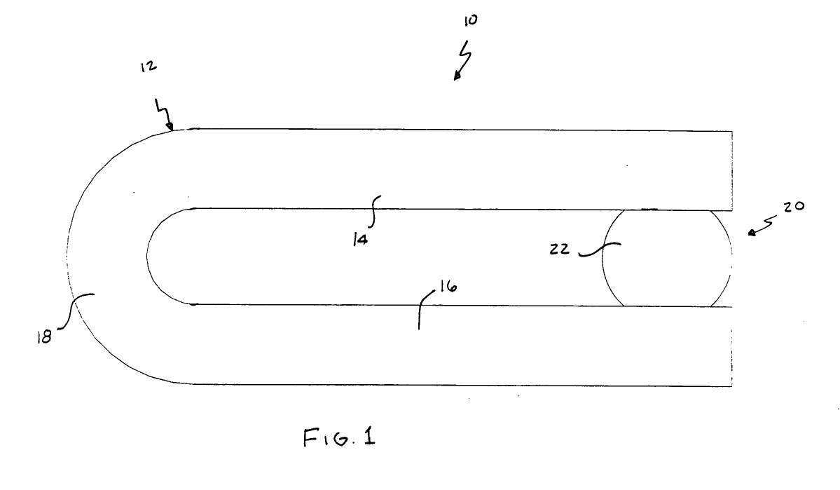

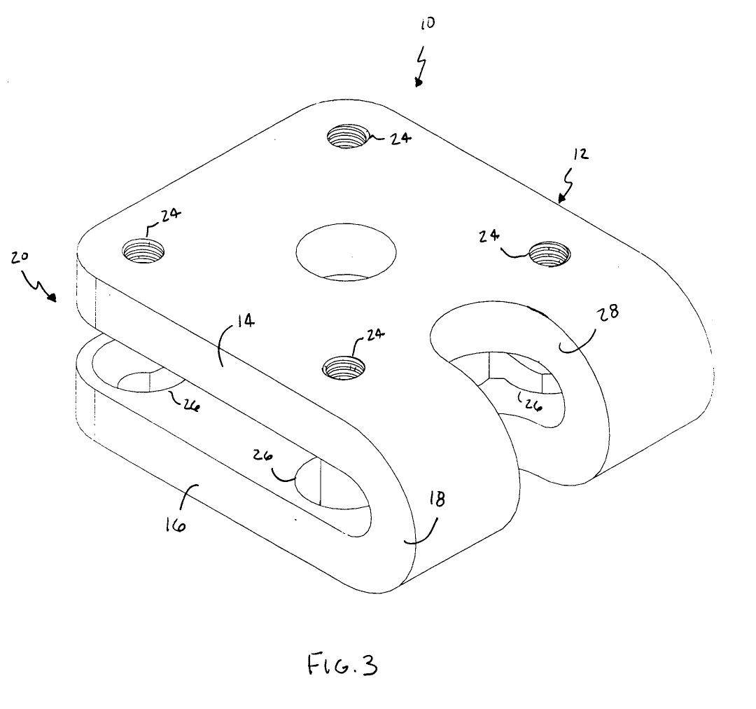

FIGS. 1-4 illustrate an exemplary embodiment of a modular stance flexion component 10 according to an exemplary embodiment of the present invention. The component generally includes a C-shaped body 12 of a strong, lightweight and resilient material such as a carbon fiber material. The body 12 is essentially a sheet of such material folded upon itself in the shape of a C to provide a pair of horizontally extending top and bottom extensions 14, 16 emanating from an anterior curved end 18. Positioned between the two extensions at the posterior open end 20 of the C-shaped body is a sphere 22 of rubber-like material providing a bias between the extensions 14, 16 at the open end 20. In this exemplary embodiment, the rubber-like material may be a Santaprene material and the sphere 22 diameter is approximately 3/8″ or more. The sphere 22 may be retained in this position by any manner of known mechanical and/or chemical couplings/fasteners such as, for example and without limitation, providing grooves on the sphere 22 for mating with complementary ribs on the top/bottom extensions 14, 16 or providing concave depressions in the top/bottom extensions 14, 16 for seating the sphere 22 therein.

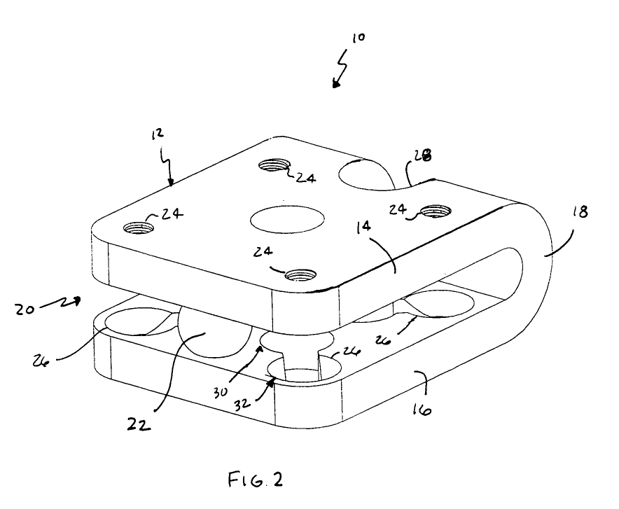

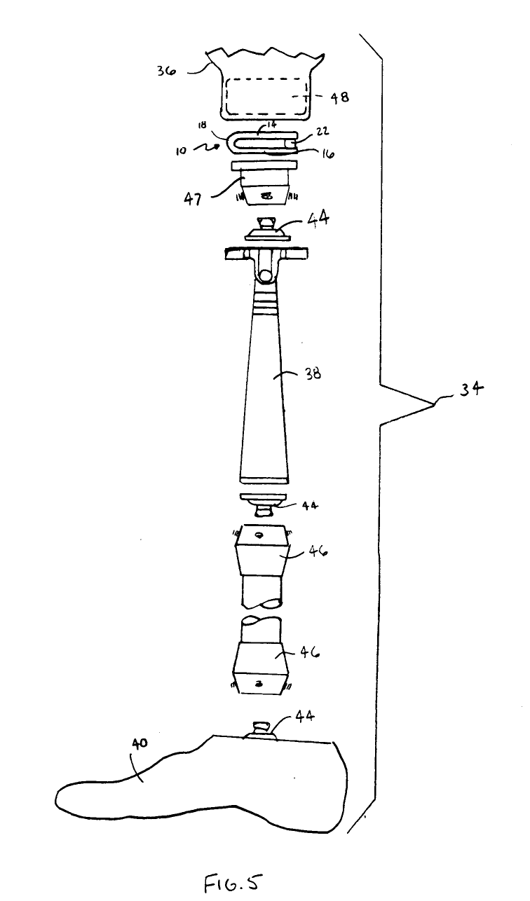

Referring to FIGS. 2-4, the top extension 14 includes a set of four tapped holes 24 adapted to receive threaded screws extending from a corresponding proximal prosthetic limb component in a traditional four-hole pattern. Likewise, the bottom extension 16 includes a set of four through-holes 26 adapted to receive and seat therewithin flat head socket cap screws for screwing into tapped holes extending into a distal prosthetic limb component in a traditional four-hole pattern. Consequently, the tapped holes 24 and through holes 26 are coupling expedients for coupling the modular component 10 between a pair of prosthetic limb components. This will be described in further detail below with respect to FIG. 5. It is also within the scope of the invention to provide more than one set or pattern of the four tapped holes 24 or through-holes 26 in an extension 14, 16, for additional flexibility in coupling to various prosthetic limb components. Furthermore, the modular component 10 is able to be flipped over if desired (i.e., the top extension becomes the bottom extension adapted to be coupled to a distal prosthetic limb component and vice-versa), while keeping the curved end 18 in an anterior orientation. As will be described further below with respect to FIG. 5, this ability of the modular component 10 to be flipped over adds additional flexibility for interconnecting a broader range of prosthetic limb components.

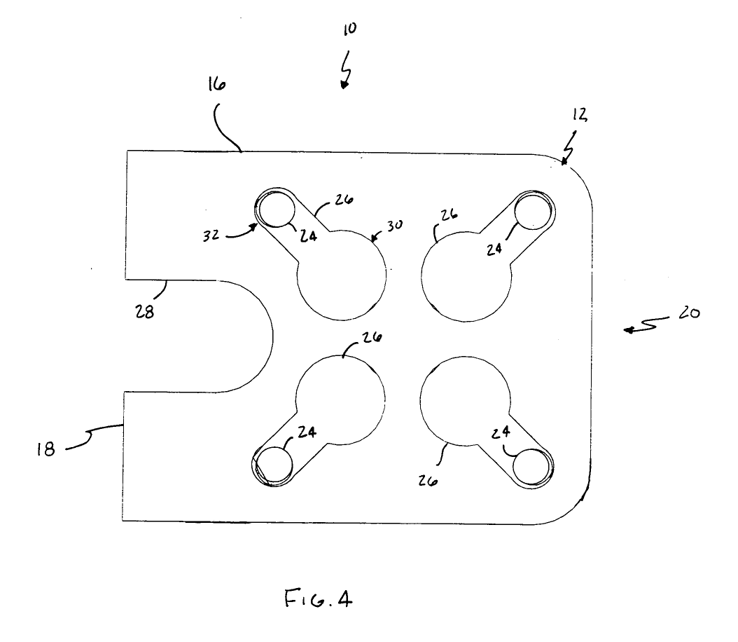

Referring to FIGS. 2 and 4, the through holes 26 include a cylindrical portion 30 and a concave portion 32. The cylindrical portion 30 has a diameter sufficient to allow the head of the flat head socket cap screw to extend therein from the outer surface. Thus, after the head of the flat head cap screw is inserted therethrough it is drawn over to the concave portion 32 where the concavity seats the conical head of the flat head screw therein while allowing the threaded shaft of the screw to extend outwardly through the narrow opening in the conical portion 32. Once seated, the heads of the flat head socket cap screws may be accessed by a narrow screw-driver extending through a corresponding one of the tapped holes 24 in the opposing extension 14.

As further shown in FIG. 2-4, the curved end 18 of the C-shaped body 12 includes a notch 28 extending vertically there through to augment flexibility at the pivot point formed by the C-shaped end 18.

Referring now to FIG. 5, a prosthetic limb 34 is shown in exploded view, having the modular stance flexion component 10 of FIGS. 1-4 coupled between a prosthetic limb socket component 36 and a prosthetic limb knee chassis 38. Specifically, in this embodiment, the top extension 14 is coupled directly to the socket component 36 and the bottom extension 16 is coupled directly to a pyramid receiver 47, which in turn is coupled directly to a pyramid component 44 coupled to, or extending from the knee chassis 38. The prosthetic limb 34 also includes a prosthetic foot component 40 coupled to a distal end of a pylon component 42 by a pyramid 44 and a pyramid receiver 46. The pylon component 42 is, in turn, coupled to the distal end of the knee chassis component 38 by a pyramid 44 and pyramid receiver 46. Carried within the distal end of the prosthetic socket component is a shuttle lock component 48.

The shuttle lock component 48 may be a top-loaded PDI Xtreme® suspension lock in which threaded screws (not shown) extend distally from the lock 48 through the distal end of the prosthetic limb socket 36 and into the threaded holes 24 of the top extension 14 modular stance flexion component 10. As discussed above, flat head socket cap screws (not shown) extend from the distal end of the modular stance flexion component into correspondingly threaded holes in the base of the pyramid receiver 47.

As discussed above, it is possible to ‘flip’ the modular stance flexion component 10 over such that the extension 14 becomes the bottom or distal extension and such that the extension 16 becomes the top or proximal extension, so long as the pivot point provided by the curved end 18 remains positioned on the anterior side of the modular component. With the embodiment illustrated in FIG. 5, if the modular stance flexion component 10 were flipped over in this manner, the lock 48 would then be a standard bottom-loaded PDI Xtreme® lock that receives the shafts of flat head socket cap screws extending upwardly from the now-top extension 16. And the pyramid receiver 47 would have countersunk through holes for seating screws therein, where the shafts of such screws would extend upwardly into the threaded holes 24 of the now-bottom extension 14.

In use, during ambulation, the extensions 14 and 16 will flex together at their respective posterior ends, pivoting about a pivot point formed by the curved anterior end 18, during heel strike or any other weight-bearing circumstance. The sphere 22 will absorb shocks during such weight-bearing event and will also provide a biasing element to help bias the extensions 14 and 16 back apart upon the weight-bearing event ending. Certainly, an array of durometer settings may be provided for these spheres 22 to provide stance flexion adjustability.

It is also within the scope of the invention to provide a hyperextension limiting element, such as a posterior strap (not shown) which will prevent the flexion stance component 10 from hyperextending (i.e., the posterior ends of the extensions 14, 16 pivoting apart from each other too far). This limitation on hyperextension may be also facilitated by a mechanical linkage extending through the sphere 22 and into the top and bottom extensions. It should also be apparent to those of ordinary skill that the bias and shock absorbing affects provided by the sphere 22 may be provided by other elements such as springs, and/or alternate shapes such as short cylinders.

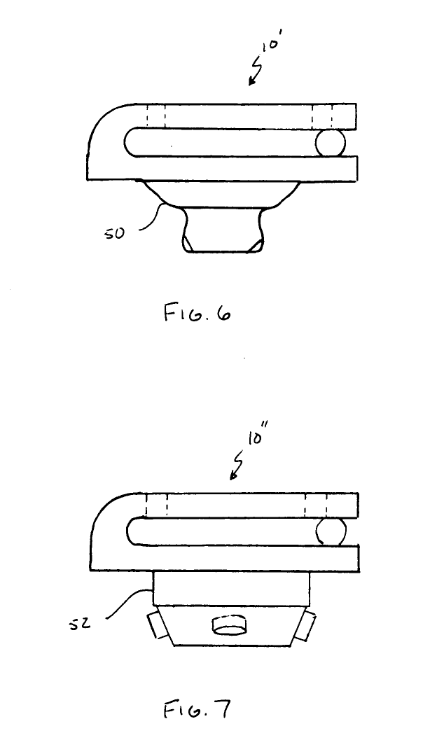

FIG. 6 provides an alternate embodiment 10′ of the modular stance flexion component incorporating a pyramid component 50 into one of the extensions. This pyramid component may be both rotatably and laterally adjustable using mechanisms similar to those as shown in U.S. Pat. Nos. 6,033,440 and 6,231,618.

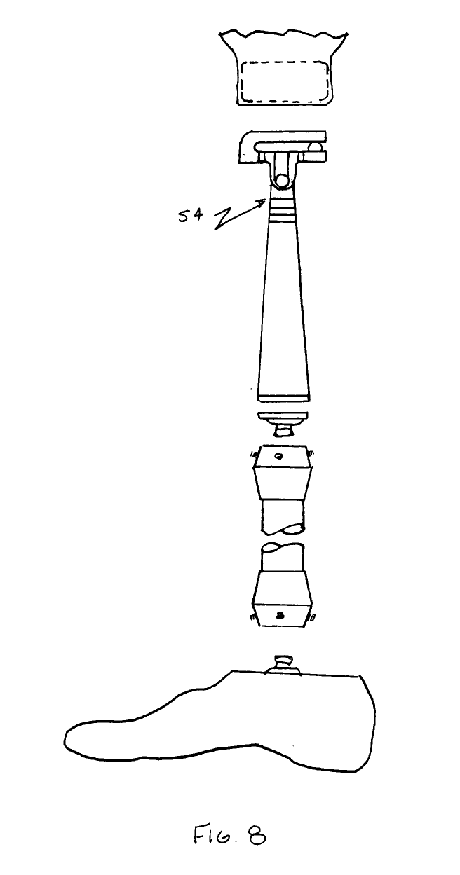

FIG. 7 illustrates a second alternate embodiment of a modular stance flexion component 10″ which includes a pyramid receiver component integrated into one of the extensions as a coupling expedient. As with the pyramid component 50 of FIG. 6, the pyramid coupling component 52 may also be rotatably and laterally adjustable using mechanisms similar to those as shown in U.S. Pat. No. 6,458,163.

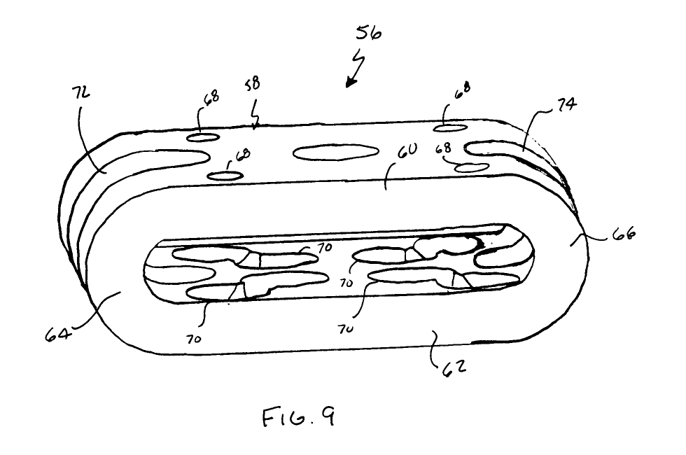

FIG. 8 illustrates a prosthetic limb component in which the C-shaped body of the modular stance flexion component of FIGS. 1-4 is integrated into the proximal end of a knee chassis 54. In this embodiment, the bottom extension is integrated into a proximal end of the knee chassis, while the remaining components remain substantially unchanged.

FIG. 9 illustrates an embodiment of a modular vertical shock reducing component 56 built upon the concepts of the modular stance flexion component 10 as described in FIGS. 1-4. The modular vertical shock reducing component 56 is essentially a body of carbon fiber or some other lightweight, high strength material formed in the shape of a “squashed O” and adapted to be coupled between a pair of prosthetic limb components. Essentially, the body 58 includes a pair of opposing, substantially flat horizontally extending top/bottom ends 60, 62 interconnected by a pair of opposing, substantially curved anterior/posterior ends 64, 66. Each of the flat horizontally extending top/bottom ends 60, 62 of the body include coupling expedients such as, for example, threaded holes 68 or through-holes 70 as described in the above embodiments. Each of the substantially curved anterior/posterior ends 64, 66 also includes a vertically extending notch 72, 74 to augment flexibility of the curved ends.

In use, the component 56 is coupled vertically between a pair of prosthetic limb components such that it will compress under weight bearing loads. Similar to the modular stance flexion components described above, the modular vertical shock reducing component 56 will provide shock absorption during ambulation. As with the above embodiment, the modular component 56 is able to be flipped over if desired (i.e., the top end becomes the bottom end adapted to be coupled to a distal prosthetic limb component and vice-versa).

Furthermore, similar to the modular stance flexion component described above with respect to FIGS. 1-4, the modular vertical shock reducing component 56 may incorporate a pyramid component and/or a pyramid receiver component into one or both of the substantially flat horizontally extending top/bottom ends 60, 62; and, further, one of the substantially flat horizontally extending top/bottom ends 60, 62 may be integrated within the proximal end of a knee-shin system (or any other prosthetic limb component) similar to the embodiment of the stance flexion component shown in FIG. 8.

Following from the above detailed description, it will be apparent to those of ordinary skill in the art that, while the apparatuses and processes herein described constitute exemplary embodiments of the present invention, it is understood that the invention is not limited to these precise apparatuses and processes and that changes may be made therein without departing from the scope of the invention as claimed or as illustrated by the various aspects of the present invention set forth in the summary. Additionally, it is to be understood that the invention is defined by the claims and it is not intended that any limitations or elements describing the exemplary embodiments set forth herein are to be incorporated into the meanings of the claims unless such limitations or elements are explicitly listed in the claims. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

1. A prosthetic leg assembly comprising:
a prosthetic socket component for receiving a patient's residual limb therein;
a prosthetic knee component;
a pylon assembly coupled to a distal end of the prosthetic knee component;
a prosthetic foot coupled to a distal end of the pylon assembly; and
a modular component selectively interconnected between the prosthetic knee component and the prosthetic socket component, the modular component providing or augmenting stance flexion approximate the prosthetic knee component.

2. The prosthetic leg assembly of claim 1, wherein the modular component comprises:
a proximal end coupled to a proximal prosthetic limb component;
a distal coupled to a distal prosthetic limb component;
an anterior pivot point operatively provided between the proximal and distal ends, which allows the proximal and distal prosthetic limb components to pivot with respect to each other; and
a bias operatively provided between the proximal and distal ends, which allows the proximal and distal prosthetic limb components to flex towards each other approximate the posterior ends thereof at least under weight bearing forces and/or heel strike.

3. The prosthetic leg assembly of claim 2, wherein:
the distal prosthetic limb component is one of, (a) a component coupled to the prosthetic knee component, (b) a component of an assembly coupled to the prosthetic knee component, and (c) the prosthetic knee component; and
the proximal prosthetic limb component is one of, (a) a component coupled to the prosthetic socket component, (b) a component of an assembly coupled to the prosthetic socket component, and (c) the prosthetic socket component.

4. The prosthetic leg assembly of claim 2, wherein the modular component comprises:
a substantially C-shaped body of flexible material, having a pair of substantially horizontal extensions emanating from a curved end to an open end, the extensions of the C-shaped body including a proximal extension and a distal extension and each extension including means for coupling to other prosthetic limb components; and
a shock-absorber operatively provided between the proximal and distal extensions;
the curved end of the C-shaped body positioned approximate the anterior of the prosthetic knee component and the open end of the C-shaped body positioned approximate the posterior of the prosthetic knee component.

5. The prosthetic leg assembly of claim 4, wherein the shock-absorber is positioned approximate the open end of the C-shaped body.

6. The prosthetic leg assembly of claim 4, wherein the shock-absorber includes a rubber-like sphere.

7. The prosthetic leg assembly of claim 4, wherein the C-shaped body is formed from a carbon fiber material.

8. The prosthetic leg assembly of claim 4, wherein the modular component further comprises a retainer coupled between the proximal and distal extensions to limit posterior outward expansion of the C-shaped body.

9. The prosthetic leg assembly of claim 1, wherein the modular component comprises:
a substantially C-shaped body of flexible material, having a pair of substantially horizontal extensions emanating from a curved end to an open end, the extensions of the C-shaped body including a proximal extension and a distal extension and each extension including means for coupling to other prosthetic limb components; and
a shock-absorber operatively provided between the proximal and distal extensions;
the curved end of the C-shaped body positioned approximate the anterior of the prosthetic knee component and the open end of the C-shaped body positioned approximate the posterior of the prosthetic knee component.

10. A modular prosthetic limb stance flexion component comprising:
a proximal end including means for selectively coupling to a proximal prosthetic limb component;
a distal end including means for selectively coupling to a distal prosthetic limb component;
an anterior pivot point operatively provided between the proximal and distal ends, which allows the proximal and distal prosthetic limb components to pivot with respect to each other; and
a bias operatively provided between the proximal and distal ends, which allows the proximal and distal prosthetic limb components to flex towards each other approximate the posterior ends thereof at least under weight bearing forces and/or heel strike.

11. A modular prosthetic limb stance flexion component comprising:
a substantially C-shaped body of flexible material, having a pair of substantially horizontal extensions emanating from a curved end to an open end, the extensions of the C-shaped body including a proximal extension and a distal extension and each extension including means for coupling to other prosthetic limb components; and
a shock-absorber operatively provided between the proximal and distal extensions;
the curved end of the C-shaped body adapted to be positioned approximate the anterior of a prosthetic knee component and the open end of the C-shaped body adapted to be positioned approximate the posterior of the prosthetic knee component.

12. The prosthetic leg assembly of claim 11, wherein the shock-absorber is positioned approximate the open end of the C-shaped body.

13. The prosthetic leg assembly of claim 11, wherein the shock-absorber includes a rubber-like sphere.

14. The prosthetic leg assembly of claim 11, wherein the C-shaped body is formed from a carbon fiber material.

15. The prosthetic leg assembly of claim 11, wherein the modular component further comprises a retainer coupled between the proximal and distal extensions to limit posterior outward expansion of the C-shaped body.

16. A method for providing or augmenting stance flexion in a prosthetic limb comprising the step of selectively coupling a modular stance flexion component between a prosthetic limb socket component and a prosthetic limb knee component.

17. The method of claim 16, wherein the modular stance flexion component includes:
a proximal end;
a distal end including means for selectively coupling to a distal prosthetic limb component;
an anterior pivot point operatively provided between the proximal and distal ends, which allows the proximal and distal prosthetic limb components to pivot with respect to each other; and
a bias operatively provided between the proximal and distal ends, which allows the proximal and distal prosthetic limb components to flex towards each other approximate the posterior ends thereof at least under weight bearing forces and/or heel strike; and
the step of selectively coupling includes a step of selectively coupling the proximal end of the modular stance flexion component to a proximal prosthetic limb component and a step of selectively coupling the distal end of the modular stance flexion component to a distal prosthetic limb component.

18. A prosthetic knee assembly comprising:
a prosthetic knee chassis including a proximal end; and
a flexible flange extending from the proximal end of the knee chassis forming a substantially C-shaped channel between the flexible flange and the proximal end of the knee chassis, the C-shaped channel having a posterior-facing open end, and the flexible flange including means for coupling to proximal prosthetic limb components.

19. The prosthetic knee assembly further comprising a shock-absorber operatively provided between the flexible flange and the proximal end of the knee chassis.

20. A modular prosthetic limb component adapted to be selectively interconnected between two prosthetic limb components comprising a body of flexible material substantially in the shape of a vertically compressed-O, having a pair of opposing, substantially flat horizontally extending top and bottom ends interconnected by a pair of opposing, substantially curved anterior and posterior ends, each of the flat horizontally extending top and bottom ends including means for removably coupling the respective top and bottom ends to a respective one of the two prosthetic limb components, whereby each of the curved anterior and posterior ends allow the top and bottom ends to flex together at least upon moments of impact such as heel strike.

21. The modular prosthetic limb component of claim 20, wherein the body is formed from a carbon fiber material.
